(19) United States
(12) Patent Application Publication
(10) Pub. No.: US 2004/0137080 A1
(43) Pub. Date: Jul. 15, 2004

(54) Multivitamin regimen for renal patients

(76) Inventor: Henry D. Cremisi, Charlotte, NC (US)

Correspondence Address: MCDERMOTT WILL & EMERY, 600 13TH STREET, N.W., WASHINGTON, DC, 20005-3096, US

(21) Appl. No.: 10/340,837

(22) Filed: Jan. 13, 2003

(51) Int. Cl. A61K 35/78; A61K 33/04; A61K 31/714; A61K 31/50; A61K 31/525; A61K 31/51

(52) U.S. Cl. 424/702; 424/750; 514/251; 514/276; 514/52; 514/250; 514/356; 514/350; 514/561; 514/393; 514/474

(57) ABSTRACT

A vitamin and mineral supplement regimen for dialysis patients. The regimen comprises first and second formulations. The first formulation contains selenium, and no zinc, and is administered on days of dialysis. The second formulation contains zinc, and no selenium, and is administered on days between dialysis. In addition, the second formulation contains more vitamin C and folic acid than the first formulation.

### FIELD OF THE INVENTION

The invention pertains to a vitamin and mineral supplement regimen for dialysis patients and method for administering the same. The regimen prescribes a vitamin and mineral composition for days wherein the patient is undergoing dialysis treatment and a different vitamin and mineral composition for days between dialysis treatments. The regimen also is a benefit in that it will lower serum homocystine levels.

### BACKGROUND OF THE INVENTION

The kidneys perform a variety of physiological functions—excretory, metabolic, regulatory and endocrine. They control fluid and electrolyte homeostasis and excretion of nitrogenous wastes. They also play a role in arterial pressure regulation by secreting vasoactive substances such as renin, by secreting erythropoietin, which stimulates red blood cell production and by producing 1,25-dihydroxy vitamin D₃, which is the active form of vitamin D. Any of these functions can be impaired by renal disease. Thus, the loss of renal function affects multiple body organ systems to a significant degree.

Renal disease may be classified into two categories. The first category is acute renal failure. This disease is characterized by a generally reversible sudden reduction or cessation of renal function. The etiology of the renal dysfunction would include immunologic (i.e. lupus), homodynamic (i.e., acute tubular necrosis), obstructive (i.e., bladder outlet obstruction), infectious (pyelonephritis), allergic (i.e., interstitial nephritis), vascular (i.e., cholesterol embolization), and/or drug induced (contrast nephropathy) components. Typically, this type of renal failure does not require dialysis, but can be managed with diet, medication, and intensive monitoring. Dialysis is indicated and initiated in this setting when the fluid and electrolyte imbalances imperil the patient. The second category is chronic renal failure, which reflects a progressive irreversible loss of renal function. Chronic renal failure is generally caused by immunological disorders such as glomerulonephritis and metabolic disorders such as diabetes mellitus and hypertension, in addition to all the etiologies that caused acute renal failure. A progressive deterioration of nephrologic function in chronic renal failure can lead to end-stage renal failure, which is a condition where the kidneys can no longer provide adequate renal excretory and regulatory functions. Such failure results in toxins accumulating in the body, such as acid, phosphorous, urea, and creatinine. This inability to excrete adequate fluid facilitates edema and volume overload with the potential for respiratory compromise. The accumulation of toxins results in acidosis and uremia. The excess fluid and toxins must be removed by dialysis.

There are two types of dialysis treatment. The first type of dialysis is hemodialysis, which is a process wherein toxins are removed from the blood using diffusion across a semi-permeable membrane as well as convection via ultrafiltration. Blood is removed from the patient through a permanent (surgically created arteriovenous fistula or arteriovenous graft) or temporary vascular access (a single or dual lumen catheter) inserted into the subclavian, internal jugular or femoral vein. The blood is pumped from the patient to a unit containing the semi-permeable membrane. Crystalloid (dialysate) is coursed countercurrent to the blood flow separated by a semi-permeable membrane through which waste products are exchanged by means of diffusion across a concentration gradient and excess body volume is ultra-filtered. The dialyzed blood is returned to the patient through the venous line or lumen. Patients will generally receive parenteral iron, erythropoietin, and a vitamin D3 analogue, with their dialytic therapy.

Hemodialysis patients are exposed to extremely large volumes of water. The estimated water intake of a healthy individual is 2 liter per day or 14 liters per week. A hemodialysis patient may be exposed to 350 to 500 liters of water per week, depending on their treatment time and dialysate flow rate. Municipal water supplies may contain a variety of contaminants that are toxic to hemodialysis patients. Toxic water contaminants include aluminum, calcium, chloramines, copper, fluoride, magnesium, nitrates, sodium, sulfate, zinc, as well as bacteria and endotoxin. Water purification is of paramount importance as hemodialysis patients are unable to renally excrete any contaminants taken up from the dialysate. Specific water purification processes are applied in series including reverse osmosis, deionizers, softeners, carbon adsorption, and filters. Municipal water supplies vary from location to location, even from one part of a city to another. Because of this no single water treatment system will satisfy all situations.

The second type of dialysis is peritoneal dialysis. The peritoneal dialysate (distinct in composition as compared to that used in hemodialysis) is introduced into the peritoneal cavity through a catheter. The peritoneal membrane provides a barrier that allows for waste product diffusion across a concentration gradient to the introduced dialysate. Excess bodily fluid flows via an osmotic gradient to the relatively hyperosmolar peritoneal dialysate. The dialysate is intermittently introduced into and removed from the peritoneal cavity. The transfer of diffusable solutes and water between the blood and the peritoneal cavity depends on the concentration gradient between the two fluid compartments. Peritoneal dialysis poses less hemodynamic stress than hemodialysis and is readily adaptable for home use, whereas hemodialysis patients typically are treated in a dialysis clinic or facility. Typically, hemodialysis patients undergo treatment three times a week, whereas peritoneal dialysis is a continuous process.

The pre-dialysis progression of renal disease can be constrained by managing the patient's diet. The primary goal of diet in renal disease is to control energy and nitrogen intake to optimize nutritional status. However, dietary management impacts the metabolism and utilization of many nutrients. Control of dietary protein and maintaining non-protein caloric intake are the highest priorities. Typically, the diet is used to control protein intake to minimize the accumulation of nitrogeneous waste and limit uremic symptoms while providing adequate nitrogen to prevent wasting of lean body mass. Modifications in dietary phosphorus, potassium and/or sodium intakes are also necessary to limit the accumulations of these elements in the blood or the development of hypertension. However, these dietary restrictions are generally deficient in the daily requirements for nutrients such as folic acid, the B vitamins, vitamin C, zinc and selenium. Concomitant medical therapies can yield thiamine and riboflavin deficiency. Subsequently, upon initiation of renal replacement therapy the dialysis procedure itself may remove essential vitamins, minerals and nutrients. This is superimposed on a patient population that is unlikely to be meeting their basic nutritional based upon their multiple co-morbidities.

The fact that dialysis patients have difficulty in obtaining proper dietary amounts of essential vitamins and minerals has resulted in the formulation of vitamin and nutrient supplements for renal patients. Products currently on the market include DIATX (Pamlab, LLC), RENAX Caplets (Everett Laboratories, Inc.) and NEPHROCPS (Fleming & Company). These vitamin formulations contain soluble ingredients such as folic acid, biotin, niacin, pantothenic acid, thiamine (vitamin B₁), riboflavin (vitamin B₂), pyridoxine (vitamin B₆), vitamin B₁₂ (cyanocobalamin) and vitamin C (ascorbic acid), selenium and zinc. These vitamins are taken on a daily basis to supplement vitamins and minerals lost because of a restricted diet and dialysis.

These formulations, however, do not take into account the special nutritional needs of renal patients on days when they undergo dialysis treatment (i.e., substantive folic acid losses due to it's high water solubility and patients dialytic water exposure) and days between dialysis treatments. Neither do they address the impact of vitamin-vitamin, mineral-mineral, and vitamin-mineral interactions seen at the required supplementation levels for these patients. One formulation does not fit all situations. The invention herein addresses the different nutritional needs of patients on days of dialysis and days in between dialysis.

In addition to the above, this invention is beneficial for patients diagnosed with hyperhomcysteinaemia, which is common in dialysis patients. Hyperhomcysteinaemia is a risk factor for atherosclerosis and is characterized by high serum homocysteine levels that lead to blood vessel damage. It is known that a combination of folic acid, vitamin B₆ and vitamin B₁₂ lower high serum homocysteine by converting homocysteine to methionine. Vitamin B₁₂ is necessary for taking a one-carbon unit from folic acid and delivering it to homocysteine to convert homocysteine to methionine. Vitamin B₆ is involved in a different pathway, but it is necessary for removing of excess homocysteine.

The formulations of the present invention include folic acid, vitamin B₆ and vitamin B₁₂ which are beneficial to reducing elevated serum homocysteine levels. The formulations have the further advantage of not including antioxidants such as iron or copper which are known to destroy some of Vitamin B₁₂ and some of the folic acid.

### SUMMARY OF THE INVENTION

The present invention is directed to a vitamin and mineral supplement regimen for dialysis patients. An object of the invention is to provide a weekly regimen or kit of vitamin supplements for dialysis patients to supplement their nutritional needs. It a further object of the invention to provide a vitamin and mineral supplement for days the dialysis patient undergoes dialysis and a vitamin and mineral supplement for days between dialysis. It is still a further aspect of the present invention is to provide a method of administering a multiple vitamin supplement composition for lowering high serum homocysteine levels to protect against the incidence of heart attack and other cardiovascular related disorders. These and other objects and characteristics of the present invention will become apparent in the detailed description of the invention.

### DETAILED DESCRIPTION OF THE INVENTION

This invention relates to a vitamin and mineral supplement regimen for renal patients who are being treated by dialysis. The regimen consists of a vitamin and mineral supplement formulation for days when the patient is undergoing dialysis and a second vitamin and mineral supplement formulation for days between dialysis treatments. The formulations are also beneficial to lower serum homocysteine levels.

The formulations of the prior art do not take into account the special nutritional needs of patients on days when patients undergo dialysis treatment and days between dialysis treatment. One formulation does not fit all situations. In particular, the nutritional needs of the patient on days between dialysis treatments are different from those days when dialysis is required. Specifically, when the patient undergoes dialysis, a significant amount of selenium and vitamin C are lost during dialysis therapy and therefore must be supplemented. As for zinc, it is not necessary to supplement this mineral because it is usually present as a contaminate in the dialysis fluids. Therefore, it is only necessary to supplement zinc on days between dialysis.

The vitamin and mineral supplements for renal patients, especially those in renal failure who must be treated by dialysis is different from normal recommended dietary allowances (RDA). This is due to dialysis which removes vitamins and minerals from the blood as well as the dietary restrictions imposed on renal patients to control potassium and phosphorous levels. The major factors which determines the qualify of life for a renal patient are the nutritional status of the patient when dialysis is commenced and the patient's ability to ingest and most efficiently metabolize the nutrition provided. The primary nutritional need for patient's undergoing homodialysis or peritoneal dialysis is to maintain metabolic homeostasis, i.e., normal functional indices, positive nitrogen balance and stable weight, by feeding them, either as a sole or supplemental source of nutrition, a balanced nutritional product. Dialysis patients are significantly to more likely to be at risk for malnutrition. Renal failure patients show losses, inter alia, in biotin, folic acid, niacin, pantothenic acid, thiamin, pyridoxine, vitamin C and selenium after dialysis. The vitamin and mineral regimen of the present invention is designed to supplement the RDA nutritional needs of dialysis patients and also to lower the risk of vascular disease that can lead to heart attacks and other cardiovascular disorders.

Thiamine (vitamin B₁) is a coenzyme for the oxidative decarboxylation of α-ketoacids and for transketolase which is a component of the pentose phosphate pathway. The activity of thiamine is inhibited by folate deficiency and malnutrition. Chronic renal failure patients placed on a low protein diet exhibit a thiamine deficiency. The supplement formulations of the present invention include thiamin in an amount ranging from about 1 mg to about 200 mg, in particular, about 5 mg.

Riboflavin (vitamin B₂) is a component of two flavin coenzymes, namely, flavin mononucleotide (FMN) and flavin adenine dinucleotide (FAD). These flavoenzymes are involved in a number of oxidation-reduction reactions including the conversion of pyridoxine and niacin. Renal patients on a low protein diet often have a riboflavin deficiency. Thus, the supplement formulations of the present invention include riboflavin in an amount ranging from about 1 mg to about 20 mg, in particular, about 2 mg.

Niacin (vitamin B₃) includes active coenzymes nicotinamide adenine dinucleotide (NAD) and nicotinamide adenine dinucleotide phosphate (NADP). These coenzymes are involved in numerous enzymatic reactions such as glycolysis, fatty acid metabolism and steroid synthesis. Niacin is also required for the synthesis of pyroxidine, riboflavin and folic acid and may play a role in the reduction of total cholesterol. A low protein diet reduces the amount of niacin available causing a niacin deficiency in dialysis patients. The normal RDA requirement is 15-20 mg/day of niacin. Thus, in order to maintain an appropriate niacin level in dialysis patients, the amount of niacin in the formulations of the invention ranges from about 15 mg to about 50 mg, in particular, about 20 mg.

Pantothenic acid (vitamin B₅) is a component of the coenzyme A complex which is required to the synthesis of fatty acids, cholesterol, steroid hormones and neurotransmitters and which has a major rule in the acetylation and acylation of proteins. Low protein diets for renal patients provide a minimum amount of pantothenic acid. A decrease in pantothenic acid serum levels is observed in dialysis patients. The normal RDA requirements of pantothenic acid are 4 to 7 mg/day. However, it is recommended that renal failure patents be supplemented with 10 mg/day. Accordingly, the formulations of the invention include pantothenic acid in an amount ranging from about 10 mg to about 20 mg, in particular, about 10 mg.

Pyridoxine (vitamin B₆) has two active forms: pyridoxal-5′-phosphate and pyridoxamine-5′-phosphate. These are coenzymes and are essential for gluconeogenesis, niacin formation, and erythrocyte metabolism. A high incidence of pyridoxine deficiency is found in renal patients undergoing dialysis. Low protein diets have minimal amounts of pyridioxine. A deficiency in pyridoxine may be attributed to the suppressed immune function observed in chronic renal patients, as well as the increased plasma and tissue oxalate concentrations in renal failure. Also, it has been suggested that pyridoxine plays a role in homocysteinaemia. Pyridoxine is a coenzyme for both cystathionine synthase and cythionase enzymes that catalyze the formation of cystein from methionine. Homocysteine is an intermediate in this process and an elevated levels of plasma homocysteine. Since it is known that the administration of pyridioxine may reduce the levels of homosysteine, the formulation includes about 10 mg to about 200 mg of pyridioxine, in particular, about 15 mg per day.

Cyanocobalamin (vitamin B₁₂) is the pharmaceutical form of cobalamin which can be converted to active coenzymes methylcobalamin and 5′-deoxyadenosylcobalamin. These coenzymes are necessary for folic acid metabolism, conversion of coenzyme A and myelin synthesis. For example, methylcobalamin catalyzes the demethylation of a folate cofactor which is involved in DNA synthesis. A lack of demethylation may result in folic acid deficiency. A deficiency of vitamin B₁₂ has been observed in chronic renal failure patients and dialysis patients. Since vitamin B₁₂ has a role in folic acid metabolism and since folic acid plays a role in homocysteinemia, a supplement of vitamin B₁₂ may be effective to manage homocysteine levels in renal and dialysis patients. The amount of vitamin B₁₂ in the formulations of the invention range from about 10 mcg to about 1000 mcg, in particular, about 12 mcg.

Biotin acts as a coenzyme for a number of carboxylases and has an important role in gluconeogenesis and in fatty acid and amino acid metabolism. It is known that botin inhibits the effects of uremic toxins on tubulin polymerization. Also, it is known that the administration of biotin relieves the symptoms of uremic encephalopathy and neuropathy. In order to maintain adequate an adequate level of biotin in renal patients, renal patients need to be supplemented with no less than 300 mcg per day. Thus, the formulations of the invention include an amount ranging from about 300 mcg to about 1000 mcg, in particular, about 300 mcg.

The formulations of the invention do not contain antioxidants. They also differ in the amounts of vitamin C, niacin, folic acid, selenium and zinc. On days when the dialysis patient is undergoing dialysis, the patient is supplemented with selenium, but not zinc. On days between dialysis, the patient is supplemented with zinc, but not selenium. On days of dialysis, the patient is supplemented with an increased amount of folic acid and vitamin C.

Chronic renal failure patients typically have reduced levels of serum vitamin C. These reduced levels are most likely due to a low-potassium diet and decreased food intake. The low-potassium diet generally restricts fruit and vegetables which are abundant in potassium and vitamin C. The major biochemical role of vitamin C is as a cosubstrate in metal catalyzed hydroxylations. It also has antioxidant properties interacting directly with supreoxide hydroxyl radicals and singlet oxygen and provides antioxidation protection for folate and vitamin E. The recommended dietary allowance of vitamin C in non-renal patients is no more than 60 mg/day. On days between dialysis, dialysis patients need not be supplemented with more than the recommended daily allowance. Vitamin C is a water soluble vitamin and significant amounts are lost during dialysis. For this reason, the amount of vitamin C to be supplemented is higher on days a patient undergoes dialysis as opposed to days between dialysis. Accordingly, the amount of vitamin C on days between dialysis is in the range of about 50 mg to 60 mg, in particular, 60 mg. Renal patients should not be supplemented on these days no more than 60 mg because high supplementation on these days may increase the risk of oxalate formation in soft tissues. On days of dialysis treatment, the amount is in the range of about 100 to about 1000 mg, in particular, about 200 mg because of the loss due to dialysis.

Folic acid in its active form, tetrahydrofolate, is a coenzyme that is involved in the transfer of methyl groups and plays a role in DNA synthesis, purine synthesis, and amino acid synthesis, such as the conversion of glycine to serine and the transformation of homosysteine to methioine. The activation of folic acid requires vitamin B₁₂ for transmethylation of homocysteine to methionine, Vitamin B₁₂ is also necessary for folic acid delivery to tissues. The recommended daily allowance in non-renal patients is 0.4 mg. Renal patients should be supplemented with no less than 0.8 to 1.0 mg/day due to increased requirement in uremia. The metabolism of folic acid is altered by uremia and the absorption of tetrahydrofolate is impaired in chronic renal failure patients. There is a high incidence of homocysteimemia observed in chronic renal failure patients, and therefore, there is an increased risk of the renal patient developing atherosclerosis. Moreover, the diets generally prescribed for renal patients tend to be low in folic acid content and medications used by chronic renal failure patients may also inhibit the activity of folic acid. In view of this, the formulations of the invention on days of dialysis have increased supplements of folic acid ranging from about 5 mg to about 20 mg, in particular, about 10 mg. On days between dialysis, the amounts in the formulation range from about 0.4 mg to about 5 mg, in particular, about 1 mg.

Selenium is a component of the antioxidant enzyme, glutathione peroxidase, which plays a role in the control of oxygen metabolism, particularly catalyzing the breakdown of hydrogen peroxide. Glutathione peroxidase prevents the generation of free radicals and decreases the risk of oxidative damage to numerous tissues, including the vascular system. Selenium is lost during dialysis therapy and because of the low protein diet, selenium may be less than adequate to replace the selenium lost during dialysis. Decreases in serum selenium, selenium-dependent enzymes, and increased lipid peroxidation in dialysis patients and the oral or intravenous supplementation of selenium have proven to be effective in improving the selenium status and immune function of renal patients, while decreasing the levels of oxidative stress products. Therefore, on days of dialysis, the amount of supplemental selenium is in the range of 70 to 200 mcg. On days between dialysis, there is no need to supplement unless there is a deficiency is suspected.

Zinc is retained in a dialysis patient. Zinc plays a role in numerous metabolic activities such as nucleic acid production, protein synthesis and the development of the immune system. Studies have shown that dialysis patients and patients with renal failure have decreased serum levels of zinc. It is known that zinc supplementation improves a number of clinical symptoms observed in renal patients such as dygeusis, nerve conduction velocity, and impotency, and supplementation may restore impaired cell-mediated immunity and lymphocyte function. The normal daily requirement for zinc ranges from 12 to 15 mg. Renal patients may poorly compartmentalize zinc showing excessive or low values in different tissue compartments. An over supplement of zinc should be avoided. Also, dialysis fluids may be contaminated with zinc from adhesive plastic casings on dialysis coils or from galvanized pipes. For this reason, on days of dialysis, the formulations of the present invention do not contain zinc. On days between dialysis, the amount of zinc in the formulations of the invention ranges between about 1 mg and 50 mg, preferably about 5 mg, which is below the daily minimum requirement. However, should serum levels of zinc be low, then zinc should be supplemented until normalization of serum values is achieved.

A vitamin and mineral supplement regimen of the present invention comprises a first formulation and a second formulation. The formulations are administered on alternate days. In particular, a first formulation containing selenium as at least one an active ingredient, but not zinc, is administered on days of dialysis treatment while the second formulation containing zinc, but not selenium, as at least one active ingredient, on days between dialysis treatment. Neither the first nor the second formulation contains an antioxidant such as iron or copper.

The first formulation comprises the following formulation: about 1 mg to about 200 mg thiamine (vitamin B₁), about 1 mg to about 20 mg riboflavin (vitamin B₂), about 1 mg to about 50 mg niacin (vitamin B₃), about 1 mg to about 20 mg pantothenic acid (vitamin B₅), about 10 mg to about 200 mg pyridoxine (vitamin B₆), about 10 mcg to about 1000 mcg cyanocobalamin (vitamin B₁₂), about 300 mcg to about 1000 mcg biotin, about 100 mg to about 1000 mg vitamin C, about 5 mg to about 20 mg folic acid, and about 70 mcg to about 200 mcg selenium. The second vitamin composition comprises the following formulation: about 1 mg to about 200 mg thiamine (vitamin B₁), about 1 mg to about 20 mg riboflavin (vitamin B₂), about 1 mg to about 50 mg niacin (Vitamin B₃), about 1 mg to about 20 mg pantothenic acid (vitamin B₅), about 10 mg to about 200 mg pyridoxine (vitamin B₆), about 10 mcg to about 1000 mcg cyanocobalamin (vitamin B₁₂), about 300 mcg to about 1000 mcg biotin, about 50 mg to 60 mg vitamin C, about 0.4 mg to about 3 mg folic acid, and about 1 mg to about 50 mg zinc.

The formulations may optionally contain up to about 50 mcg chromium, up to about 1000 mg choline, up to about 2000 mg phosphatic acid, and up to about 2000 mg inositol. The formulation may further contain up to about 400 IU vitamin E.

In a preferred embodiment of the invention, the first formulation on days of dialysis treatment comprises the following formulation: about 5 mg thiamine (vitamin B₁), about 2 mg riboflavin (vitamin B₂), about 20 mg niacin (vitamin B₃), about 10 mg pantothenic acid (vitamin B₅), about 15 mg pyridoxine (vitamin B₆), about 12 mcg cyanocobalamin (vitamin B₁₂), about 300 mcg biotin, about 200 mg vitamin C, about 10 mg folic acid and about 100 mcg selenium. The second vitamin composition on days between dialysis comprises the following formulation: about 5 mg thiamine (vitamin B₁), about 2 mg riboflavin (vitamin B₂), about 20 mg niacin (vitamin B₃), about 10 mg pantothenic acid (vitamin B₅), about 15 mg pyridoxine (vitamin B₆), about 12 mcg cyanocobalamin (vitamin B₁₂), about 300 mcg biotin, 60 mg vitamin C, about 1 mg. folic acid, and about 5 mg zinc.

Assuming that the days for dialysis are Monday, Wednesday and Friday, the preferred weekly vitamin and mineral regimen for a dialysis patent is set forth in Table 1.

TABLE 1

| VITAMIN | SUN | | MON | | TUE | | WED | | THU | | FRI | | SAT | |
|---|---|---|---|---|---|---|---|---|---|---|---|---|---|---|
| Thiamine | 5 | mg | 5 | mg | 5 | mg | 5 | mg | 5 | mg | 5 | mg | 5 | mg |
| (Vitamin B₁) | | | | | | | | | | | | | | |
| Riboflavin | 2 | mg | 2 | mg | 2 | mg | 2 | mg | 2 | mg | 2 | mg | 2 | mg |
| (Vitamin B₂) | | | | | | | | | | | | | | |
| Niacin | 20 | mcg | 20 | mcg | 20 | mcg | 20 | mcg | 20 | mcg | 20 | mcg | 20 | mcg |
| Vitamin B₃) | | | | | | | | | | | | | | |
| Pantothenic Acid | 10 | mg | 10 | mg | 10 | mg | 10 | mg | 10 | mg | 10 | mg | 10 | mg |
| (Vitamin B₅) | | | | | | | | | | | | | | |
| Pyridoxine | 15 | mg | 15 | mg | 15 | mg | 15 | mg | 15 | mg | 15 | mg | 15 | mg |
| (Vitamin B₆) | | | | | | | | | | | | | | |
| Cyanocobalamin | 12 | mcg | 12 | mcg | 12 | mcg | 12 | mcg | 12 | mcg | 12 | mcg | 12 | mcg |
| (Vitamin B₁₂) | | | | | | | | | | | | | | |
| Biotin | 300 | mcg | 300 | mcg | 300 | mcg | 300 | mcg | 300 | mcg | 300 | mcg | 300 | mcg |
| Zinc | 5 | mg | 0 | | 5 | mg | 0 | | 5 | mg | 0 | | 5 | mg |
| Selenium | 0 | | 100 | mcg | 0 | | 100 | mcg | 0 | | 100 | mcg | 0 | |
| Folic Acid | 1 | mg | 10 | mg | 1 | mg | 10 | mg | 1 | mg | 10 | mg | 1 | mg |
| Vitamin C | 60 | mcg | 200 | mcg | 60 | mcg | 200 | mcg | 60 | mcg | 200 | mcg | 60 | mcg |

The formulations of the invention may optionally include other vitamins and minerals such as up to about 50 mcg chromium, up to about 1000 mg choline, up to about 2000 mg phosphatic acid, up to about 2000 mg inositol, and up to about 400 IU vitamin E. Chromium assists in the regulation of glucose metabolism, and is used in the synthesis of fatty acids and cholesterol, assists in the transportation of proteins lowers LDL blood levels and raised HDL blood levels. In the multi-vitamin and mineral supplement of the present invention, chromium is does in a pharmaceutically acceptable chromium compound such as chromium polynicotinate. Choline is necessary for nervous system function and brain function as well as a role in lowering homocysteine levels. It is also important for gall bladder and liver function.

The nutritional supplements of the present invention are suitably provided in any suitable dosage form known in the art. For example, the compositions are suitably incorporated into parenteral ampules for intravenous dosing, tablets, powders, granules, beads, chewable lozenges, gel capsules, liquids, or similar conventional dosage forms, using conventional equipment and techniques known in the art. Tablet and gel capsule dosage forms are preferred.

When preparing dosages forms incorporating the compositions of the present invention, the nutritional components are normally blended with conventional excipients such as binders, including gelatin, pregelatinzed starch, and the like; lubricants such as hydrogenated vegetable oil, stearic acid and the like; diluents such as lactose, mannose, and sucrose; disintegants such as carboxymethyl cellulose and sodium starch glycolate; suspending agents such as providone, polyvinyl alcohol, and the like; absorbents, such as silicon dioxide; preservative such as methylparabenzene, propylparabenzene and sodium benzoate; surfactants, such as sodium lauryl sulfate, polysorbate 80 and the like; and colorants.

For preparing the composition from the compounds described by this invention, inert, pharmaceutically acceptable carriers are used which are either solid or liquid form. Solid form preparations include powders, tablets, dispersible granules, gel capsules, and cachets. A solid carrier is suitably one or more substances which may also act as diluents, flavoring agents, solubilizers, lubricants, suspending agents, binders or tablet disintegrating agents. The solid carrier material also includes encapsulating material. In powders, the carrier is finely divided active compounds. In the tablet, the active compound is mixed with the carrier having the necessary binding properties in suitable proportions and compacted in the shape and size desired. Suitable solid carriers include, but are not limited, to magnesium carbonate, magnesium stearate, talc, sugar, lactose, pectin, dextrin, starch, gelatin, tragacanth, methylcellulose, sodium carboxymethyl-cellulose, a low melting wax, cocoa butter and the like. The term preparation is intended to include the formulation of the active compounds with encapsulating material as the carrier providing a capsule in which the active component (with or without other carriers) is surrounded by carrier, which is thus in association with it. Tablets, powders, cachets, and gel capsules may be used in a solid dosage form suitable for oral administration.

Liquid form preparations include solutions, suspensions, and emulsions. Aqueous solutions suitable for oral use are prepared by dissolving the active component in water or other suitable liquid and adding suitable colorants, flavors, stabilizing agents, and thickening agents as desired. Aqueous solutions suitable for oral use may also be made by dispersing the finely divided active component in water or other suitable liquid with viscous material, such as natural or synthetic gums, resins, methylcellulose, sodium carboxymethylcellulose, and other suspending agents known in the art.

Also included are solid form preparations which are intended to be converted, shortly before use, to liquid form preparations for either oral or parental administration. Such liquid forms include solutions, suspensions and emulsions. These particular solid form preparations are provided in unit dose form and as such are used to provide a single liquid dosage unit. Alternatively, sufficient solid preparation may be provided so that the after conversion to liquid form, multiple individual liquid doses may be obtained by measuring predetermined volumes of the liquid form preparation as with a syringe, teaspoon, or other volumetric contained.

The solid and liquid forms may contain, in addition to the active material, flavorants, colorants, stabilizers, buffers, artificial and natural sweeteners, dispersants, thickeners, solubilizing agents and the like. The liquid utilized for preparing the liquid form preparation is suitably water, isotonic water, ethanol, glycerin, propylene glycol and the like as well as combinations thereof. The liquid utilized will be chosen with regard to the route of administration.

Preferably, the preparations are unit dosage form. In such form, the preparation is subdivided into therapeutic unit dosage amounts containing appropriate quantities of the active components. The therapeutic unit dosage form can be a packaged preparation such as packaged tablets or gel capsules, or a kit including a week supply or multiple week supply of tablets or capsules. The therapeutic unit dosage can be a capsule, cachet, or tablet itself or it can be the appropriate number of any of these in packaged form.

The present invention is further exemplified in the following examples. It is understood that the examples are only for illustrative purposes wherein the claims set forth the scope of the present invention.

### EXAMPLE 1

The first formulation, for use on days of dialysis, is prepared by mixing the following active ingredients: 5 mg thiamine hydrochloride (HCl), 2 mg riboflavin, 20 mg nicotinic acid, 15 mg pyridoxine hydrochloride (HCl), 12 mcg cyanocolalamine, 10 mg folic acid, 300 mcg botin, 10 mg D-calcium pantothenate, 200 mg ascorbic acid and 100 mcg 1-selenomethionine. To this mixture the following inactive ingredients are added: 300 mg larch arabinogalactans croscarmellose sodium, 200 mg microcrystalline cellulose, 100 mg magnesium stearate, 50 mg beta carotene, 5 mg of a dye and vanilla flavoring and 226 mg lecithin. The resultant mixture is compressed, by conventional techniques, into a tablet.

### EXAMPLE 2

The second formulation, for use on days between dialysis treatment, is prepared by mixing the following active ingredients: 5 mg thiamine hydrochloride (HCl), 2 mg riboflavin, 20 mg nicotinic acid, 15 mg pyridoxine hydrochloride (HCl), 12 mcg cyanocolalamine, 1 mg folic acid, 300 mcg botin, 10 mg D-calcium pantothenate, 60 mg ascorbic acid and 5 mg zinc gluconate. To this mixture the following inactive ingredients are added: 300 mg larch arabinogalactans croscarmellose sodium, 200 mg microcrystalline cellulose, 100 mg magnesium stearate, 50 mg beta carotene, 5 mg of a dye and vanilla flavoring and 226 mg lecithin. The resultant mixture is compressed, by conventional techniques, into a tablet.

While this invention has been described with reference to several preferred embodiments, it is contemplated that various alterations and modifications thereof will become apparent to those skilled in the art upon a reading of the preceding detailed description. It is therefore intended that the following appended claims be interpreted as including all such alterations and modifications as fall within the true spirit and scope of this invention.

What is claimed is:

1. A vitamin and mineral supplement regimen for administration to a human comprising a first formulation and a second formulation, the second formulation administered on alternate days of administering the first formulation, the first formulation containing selenium as at least one an active ingredient, but not zinc, and the second formulation containing zinc, but not selenium, as at least one active ingredient, neither the first nor the second formulation containing an antioxidant.

2. The vitamin and mineral supplement regimen of claim 1, wherein the first and second vitamin compositions further contains one or more active ingredients selected from the group consisting of thiamine (vitamin B₁), riboflavin (vitamin B₂), niacin (vitamin B₃), pantothenic acid (vitamin B₅), pyridoxine (vitamin B₆), cyanocobalamin (vitamin B₁₂), biotin vitamin C, folic acid, and selenium as active ingredients.

3. The vitamin and mineral supplement regimen of claim 2, wherein the amounts of folic acid and vitamin C in the first formulation are higher than the amounts of folic acid and vitamin C in the second formulation.

4. The vitamin and mineral supplement regimen of claim 1, wherein the first formulation contains about 70 mcg to about 200 mcg selenium.

5. The vitamin and mineral supplement regimen of claim 1, wherein the second formulation contains about 1 mg to about 50 mg zinc.

6. The vitamin and mineral supplement regimen of claim 1 or 2, wherein the first formulation contains about 70 mcg to about 200 mcg selenium and the second formulation contains about 1 mg to about 50 mg zinc.

7. The vitamin and mineral supplement regimen of claim 2, wherein the first and second formulations further contain as active ingredients up to about 50 mcg chromium, up to about 1000 mg choline, up to about 2000 mg phosphatic acid, up to about 2000 mg inositol and up to about 400 IU vitamin E.

8. The vitamin and mineral supplement regimen of claim 1, wherein the first formulation comprises the following active ingredients: about 1 mg to about 200 mg thiamine (vitamin B₁), about 1 mg to about 20 mg riboflavin (vitamin B₂), about 1 mg to about 50 mg niacin (vitamin B₃), about 1 mg to about 20 mg pantothenic acid (vitamin B₅), about 10 mg to about 200 mg pyridoxine (vitamin B₆), about 10 mcg to about 1000 mcg cyanocobalamin (vitamin B₁₂), about 300 mcg to about 1000 mcg biotin, about 100 mg to about 1000 mg vitamin C, about 5 mg to about 20 mg folic acid, and about 70 mcg to about 200 mcg selenium.

9. The vitamin and mineral supplement regimen of claim 1, wherein the second formulation comprises the following active ingredients: about 1 mg to about 200 mg thiamine (vitamin B₁), about 1 mg to about 20 mg riboflavin (vitamin B₂), about 1 mg to about 50 mg niacin (Vitamin B₃), about 1 mg to about 20 mg pantothenic acid (vitamin B₅), about 10 mg to about 200 mg pyridoxine (vitamin B₆), about 10 mcg to about 1000 mcg cyanocobalamin (vitamin B₁₂), about 300 mcg to about 1000 mcg biotin, about 50 mg to 60 mg vitamin C, about 0.4 mg to about 3 mg folic acid, and about 1 mg to about 50 mg zinc.

10. The vitamin and mineral supplement regimen of claim 1, wherein the first formulation comprises the following active ingredients: about 5 mg thiamine (vitamin B₁), about 2 mg riboflavin (vitamin B₂), about 20 mg niacin (vitamin B₃), about 10 mg pantothenic acid (vitamin B₅), about 15 mg pyridoxine (vitamin B₆), about 12 mcg cyanocobalamin (vitamin B₁₂), about 300 mcg biotin, about 200 mg vitamin C, about 10 mg folic acid, and about 100 mcg selenium.

11. The vitamin and mineral supplement regimen of claim 1, wherein the second vitamin composition comprises the following active ingredients: about 5 mg thiamine (vitamin Be), about 2 mg riboflavin (vitamin B₂), about 20 mg niacin (vitamin B₃), about 10 mg pantothenic acid (vitamin B₅), about 15 mg pyridoxine (vitamin B₆), about 12 mcg cyanocobalamin (vitamin B₁₂), about 300 mcg biotin, 60 mg vitamin C, about 1 mg folic acid, and about 5 mg zinc.

12. The vitamin and mineral supplement regimen of claim 1, wherein
the first formulation comprises the following active ingredients: about 5 mg thiamine (vitamin B₁), about 2 mg riboflavin (vitamin B₂), about 20 mg niacin (vitamin B₃), about 10 mg pantothenic acid (vitamin B₅), about 15 mg pyridoxine (vitamin B₆), about 12 mcg cyanocobalamin (vitamin B₁₂), about 300 mcg biotin, about 200 mg vitamin C, about 10 mg folic acid, and about 100 mcg. selenium; and
the second formulation comprises the following active ingredients: about 5 mg thiamine (vitamin B₁), about 2 mg riboflavin (vitamin B₂), about 20 mg niacin (vitamin B₃), about 10 mg pantothenic acid (vitamin B₅), about 15 mg pyridoxine (vitamin B₆), about 12 mcg cyanocobalamin (vitamin B₁₂), about 300 mcg biotin, 60 mg vitamin C, about 1 mg folic acid, and about 5 mg zinc.

13. The vitamin and mineral supplement regimen of claim 12, wherein the dosage is in the form of a tablet or filled in a gelatin capsule.

14. A method for supplementing nutritional deficiencies in a human as result of renal disease and dialysis or to lower serum homocysteine levels or to increase bacteria production in the gut, the method comprising the step of administering a therapeutic effective amount of the vitamin and mineral regimen according to claim 1.

15. A method for supplementing nutritional deficiencies in a human as result of renal disease, dialysis, high serum homocysteine levels and low bacteria production, the method comprising the step of administering a therapeutic effective amount of the vitamin and mineral regimen according to claim 2.

16. A method for supplementing nutritional deficiencies in a human as result of renal disease, dialysis, high serum homocysteine levels and low bacteria production, the method comprising the step of administering a therapeutic effective amount of the vitamin and mineral regimen according to claim 3.

17. A method for supplementing nutritional deficiencies in a human as result of renal disease, dialysis, high serum homocysteine levels and low bacteria production, the method comprising the step of administering a therapeutic effective amount of the vitamin and mineral regimen according to claim 4.

18. A method for supplementing nutritional deficiencies in a human as result of renal disease, dialysis, high serum homocysteine levels and low bacteria production, the method comprising the step of administering a therapeutic effective amount of the vitamin and mineral regimen according to claim 5.

19. A method for supplementing nutritional deficiencies in a human as result of renal disease, dialysis, high serum homocysteine levels and low bacteria production, the method comprising the step of administering a therapeutic effective amount of the vitamin and mineral regimen according to claim 6.

20. A method for supplementing nutritional deficiencies in a human as result of renal disease, dialysis, high serum homocysteine levels and low bacteria production, the method comprising the step of administering a therapeutic effective amount of the vitamin and mineral regimen according to claim 7.

21. A method for supplementing nutritional deficiencies in a human as result of renal disease, dialysis, high serum homocysteine levels and low bacteria production, the method comprising the step of administering a therapeutic effective amount of the vitamin and mineral regimen according to claim 8.

22. A method for supplementing nutritional deficiencies in a human as result of renal disease, dialysis, high serum homocysteine levels and low bacteria production, the method comprising the step of administering a therapeutic effective amount of the vitamin and mineral regimen according to claim 9.

23. A method for supplementing nutritional deficiencies in a human as result of renal disease, dialysis, high serum homocysteine levels and low bacteria production, the method comprising the step of administering a therapeutic effective amount of the vitamin and mineral regimen according to claim 10.

24. A method for supplementing nutritional deficiencies in a human as result of renal disease, dialysis, high serum homocysteine levels and low bacteria production, the method comprising the step of administering a therapeutic effective amount of the vitamin and mineral regimen according to claim 11.

25. A method for supplementing nutritional deficiencies in a human as result of renal disease, dialysis, high serum homocysteine levels and low bacteria production, the method comprising the step of administering a therapeutic effective amount of the vitamin and mineral regimen according to claim 12.

26. The method of claims 25, wherein the method supplements nutritional deficiencies in a human as the result of renal disease.

27. The method of claims 25, wherein the method supplements nutritional deficiencies in a human as the result of dialysis and wherein the first formulation is administered on days of dialysis treatment and the second vitamin composition is administered on days between dialysis treatments.

28. A kit comprising a therapeutic dosage amount of a first formulation and a second formulation, the second formulation administered on alternate days of administering the first formulation, the first formulation containing selenium as at least one an active ingredient, but not zinc, and the second formulation containing zinc, but not selenium, as at least one active ingredient, neither the first nor the second formulation containing an antioxidant.

29. The kit according to claim 28, wherein the first formulation comprises the following active ingredients: about 1 mg to about 200 mg thiamine (vitamin B₁), about 1 mg to about 20 mg riboflavin (vitamin B₂), about 1 mg to about 50 mg niacin (vitamin B₃), about 1 mg to about 20 mg pantothenic acid (vitamin B₅), about 10 mg to about 200 mg pyridoxine (vitamin B₆), about 10 mcg to about 1000 mcg cyanocobalamin (vitamin B₁₂), about 300 mcg to about 1000 mcg biotin, about 100 mg to about 1000 mg vitamin C, about 5 mg to about 20 mg folic acid, and about 70 mcg to about 200 mcg selenium; and wherein the second formulation comprises the following active ingredients: about 1 mg to about 200 mg thiamine (vitamin B₁), about 1 mg to about 20 mg riboflavin (vitamin B₂), about 1 mg to about 50 mg niacin (Vitamin B₃), about 1 mg to about 20 mg pantothenic acid (vitamin B₅), about 10 mg to about 200 mg pyridoxine (vitamin B₆), about 10 mcg to about 1000 mcg cyanocobalamin (vitamin B₁₂), about 300 mcg to about 1000 mcg biotin, about 50 mg to 60 mg vitamin C, about 0.4 mg to about 3 mg folic acid, and about 1 mg to about 50 mg zinc.

30. The kit according to claim 28, wherein the first formulation comprises the following active ingredients: about 5 mg thiamine (vitamin B₁), about 2 mg riboflavin (vitamin B₂), about 20 mg niacin (vitamin B₃), about 10 mg pantothenic acid (vitamin B₅), about 15 mg pyridoxine (vitamin B₆), about 12 mcg cyanocobalamin (vitamin B₁₂), about 300 mcg biotin, about 200 mg vitamin C, about 10 mg folic acid, and about 100 mcg selenium; and wherein the second vitamin composition comprises the following active ingredients: about 5 mg thiamine (vitamin B₁), about 2 mg riboflavin (vitamin B₂), about 20 mg niacin (vitamin B₃), about 10 mg pantothenic acid (vitamin B₅), about 15 mg pyridoxine (vitamin B₆), about 12 mcg cyanocobalamin (vitamin B₁₂), about 300 mcg biotin, 60 mg vitamin C, about 1 mg folic acid, and about 5 mg zinc.
